(19) United States
(12) Plant Patent Application Publication
(10) Pub. No.: US 2010/0154090 P1
(43) Pub. Date: Jun. 17, 2010

(54) RASPBERRY PLANT NAMED 'PACIFIC DELUXE'

(50) ;

(75) Inventors: Mario Aguas, Freedom, CA (US); Thomas Amrhein, Watsonville, CA (US); Jose Lopez Medina, Uruapan (MX)

Correspondence Address: JONDLE & ASSOCIATES P.C., 858 HAPPY CANYON ROAD SUITE 230, CASTLE ROCK, CO, 80108, US

(73) Assignee: Pacific Berry Breeding, LLC, Salinas, CA (US)

(21) Appl. No.: 12/316,346

(22) Filed: Dec. 11, 2008

(51) Int. Cl. A01H 5/00

(52) U.S. Cl. PLT/204

(57) ABSTRACT

‘Pacific Deluxe’ is a new raspberry cultivar particularly distinguished by having large fruit throughout the season, bright color, excellent post-harvest quality and resistance to Phytophthora rubi, is disclosed.

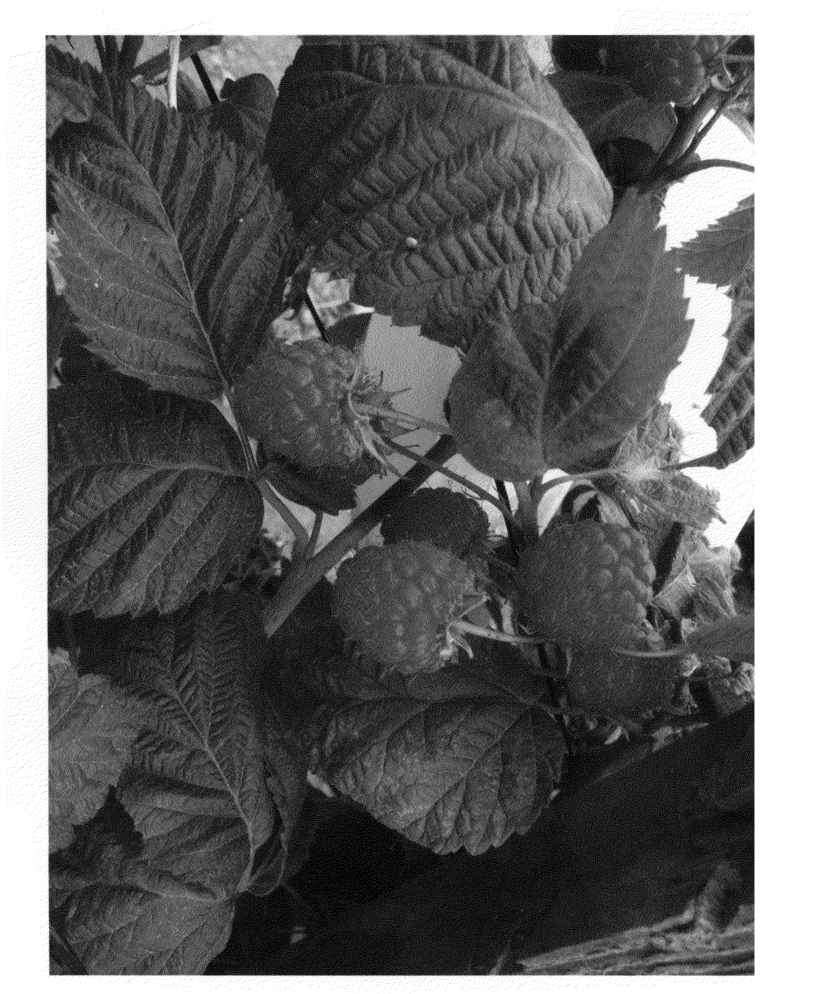

### GENUS AND SPECIES

Rubus idaeus L.

### VARIETY DENOMINATION

‘Pacific Deluxe’

### BACKGROUND OF THE NEW PLANT

This invention relates to a new and distinct variety of raspberry (Rubus idaeus L.) plant named ‘Pacific Deluxe’. ‘Pacific Deluxe’ originated as a seedling from the cross between the proprietary female raspberry parent ‘401’ (unpatented) with the proprietary male raspberry parent ‘433’ (unpatented) made in April 2003 in Oxnard, Calif.

The new cultivar was first propagated via vegetative cuttings and tissue culture in Watsonville, Calif. in June 2007 and has been asexually reproduced repeatedly by vegetative cuttings and tissue culture in Watsonville, Calif. for two generations. The present invention has been found to retain its distinctive characteristics through successive asexual propagations.

Plant Breeder's Rights for this cultivar have not been applied for prior to the filing of this application. ‘Pacific Deluxe’ has not been made publicly available more than one year prior to the filing of this application.

### SUMMARY OF THE INVENTION

The following are the most outstanding and distinguishing characteristics of this new cultivar when grown under normal horticultural practices in Watsonville, Calif.
1. Large fruit throughout the harvest season;
2. Bright color;
3. Excellent post-harvest quality; and
4. Resistance to Phytophthora rubi.

### DESCRIPTION OF THE PHOTOGRAPH

This new raspberry plant is illustrated by the accompanying photograph which shows a close-up of the plant's berry, canes and foliage from an 8 month-old plant in Watsonville, Calif. The colors shown are as true as can be reasonably obtained by conventional photographic procedures.

### DESCRIPTION OF THE NEW CULTIVAR

The following detailed description sets forth the distinctive characteristics of ‘Pacific Deluxe’. The data which define these characteristics were collected from asexual reproductions carried out in Watsonville, Calif. The plant history was taken on 1-and one-half year-old plants in August 2008. The following descriptions relate to plants grown in a field in Watsonville, Calif. Color references are to the R.H.S. Colour Chart of The Royal Horticultural Society of London (R.H.S.), 4^{th} Edition.

### DETAILED BOTANICAL DESCRIPTION

Classification:
   Family.—Rosaceae.
   Botanical.—Rubus idaeus L.
   Common name.—Raspberry.
Parentage:
   Female parent.—‘401’, a proprietary raspberry plant (unpatented).
   Male parent.—‘433’, a proprietary raspberry plant (unpatented).
Plant:
   Form and habit.—Erect growth habit; primocane fruiting; canes grow to about 1.5 m before terminating in an inflorescence.
   Height (measured from crown to apex).—120 cm to 160 cm.
   Spread (measured from leaf tip to leaf tip).—39.6 cm.
   How propagated.—Root cuttings.
   Time to initiate and develop roots.—60 days.
   Root description.—Fibrous.
   Root diameter.—0.2 cm.
Primocanes:
   Diameter.—1.01 cm.
   Length.—120 cm to 160 cm.
   Number of nodes.—25 to 35.
   Internode length.—4.8 cm.
   Number of canes/hill.—4 to 6.
   Cane color.—RHS 146D.
   Spine.—Present Spine color: RHS 59A Spines/unit area: 6 cm² Spine shape: Acuminate Spine length: 0.1 cm Spine width: 0.02 cm Spine apex: Acuminate.
Floricanes:
   Diameter.—1.0 cm to 1.2 cm.
   Length.—62 cm.
   Number of nodes.—16.
   Internode length.—5.6 cm.
   Cane color.—RHS 178A.
Leaves:
   Terminal leaflet.—Cordate, lobed.
   Arrangement.—Alternate.
   Size of terminal leaflet.—Length: 11 cm Width: 9 cm.
   Shape of apex.—Cuspidate.
   Shape of base.—Cordate.
   Margin.—Doubly-serrate.
   Texture.—Glabrous.
   Number of serrations/leaf.—97 to 109.
   Shape of serrations.—Doubly-serrate; pointed.
   Venation pattern.—Palmate.
   Venation color.—Upper surface: RHS 144B Lower surface: RHS 194B.
   Number of leaflets/leaf.—3 to 5.
   Glossiness.—Present.
   Primocane Leaves.—Color: Upper surface: RHS 137A Lower surface: RHS 189B Petiole length: 5.5 cm Petiole diameter: 0.32 cm Petiole color (upper): RHS 176C (Greyed-Orange) Petiole color (lower): RHS 144C (Yellow-Green) Rachis length: 6.0 cm Rachis diameter: 0.21 cm Rachis color (upper): RHS 176B (Greyed-Orange) Rachis color (lower): RHS 154C (Yellow-Green) Stipule length: 1.0 cm Stipule width: 0.08 cm Stipule number per leaf: 2 Stipule color: RHS 144B (Yellow-Green).
   Terminal leaflet.—Length: 11 cm Width: 9 cm Petiolule length: 2 cm Petiolule diameter: 0.2 cm Petiolule color (upper): RHS 176B (Greyed-Orange) Petiolule color (lower): RHS 154C (Yellow-Green).
   Distal lateral leaflet.—Length: 9 cm Width: 7 cm Petiolule length: 2 cm Petiolule diameter: 0.2 cm Petiolule color (upper): RHS 176B (Greyed-Orange) Petiolule color (lower): RHS 154C (Yellow-Green)
   Basal lateral leaflet.—Length: 12 cm Width: 9.5 cm Petiolule length: 2 cm Petiolule diameter: 0.2 cm Petiolule color (upper): RHS 176B (Greyed-Orange) Petiolule color (lower): RHS 154C (Yellow-Green).
Flowers:
   Bud.—Shape: Acute Length: 0.4 cm Diameter (base): 0.5 cm Diameter (tip): 0.15 cm Color: RHS 146D (Yellow-Green).
   Time of flowering (50% of plants at first flower).—Early August on primocanes.
   Size.—Length: 0.8 cm Diameter: 2 cm.
   Fragrance.—None.
   Color.—RHS 155B.
   Peduncle.—Length: 10 cm to 12 cm Diameter: 0.2 cm Color: RHS 146A Pubescence: Present.
   Petal.—Petal number per flower: 5 Petal shape: Obtuse Petal length: 0.9 cm Petal width: 0.5 cm Petal apex: Obtuse Petal base: Acute Petal margin: Entire Color (both upper and lower surfaces): RHS 155B (White).
   Flowering trusses shape.—Flattened with rounded edges.
   Sepal.—Quantity: 5 Length: 0.9 cm Width: 0.5 cm Sepal apex: Acuminate Sepal margin: Entire Color: RHS 146C.
   Pedicel.—Color: RHS 146C Length: 1.0 cm Pedicel diameter: 0.22 cm.
Reproductive Organs:
   Self Fertile.—Yes.
   Stamens (number).—64.
   Filament.—Length: 0.7 cm Diameter: 0.1 cm.
   Stamen color.—RHS 155A.
   Anthers.—Color (post dehiscence): RHS 165A Length: 0.1 cm Diameter: 0.05 cm.
   Pollen color.—RHS 155A.
   Pollen amount.—Moderate.
   Style.—Length: 0.5 cm Diameter: 0.1 cm Color: RHS 155A.
   Stigma.—Length: 0.02 cm Diameter: 0.01 cm Color: RHS 158A.
Fruit:
   Predominant shape.—Conical.
   Weight (per berry).—6 g to 8 g.
   Berry Brix.—10.0.
   Size.—Length: 2.5 cm Width: 2.0 cm.
   Receptacle.—Diameter: 1.5 cm Length: 0.9 cm Color: RHS 4D (Yellow).
   Drupelet.—Diameter: 0.3 cm Width: 0.3 cm Number: 90 to 110 Weight: 0.02 g.
   Color of mature fruit.—External: RHS 35A Internal: RHS 35C.
   Firmness of skin.—Very firm.
   Firmness of flesh.—Very firm.
   Hollow center.—Yes.
   Number of fruit per node.—4 to 8.
   Time of ripening (50% of plants with first ripe fruit).—Mid-September on primocanes.
   Time of fruiting.—Floricanes: Spring Primocanes: Late summer to fall.
   Type of bearing.—Both on previous year's canes in summer and current year's canes in autumn.
   Fruit yield.—1.5 Kg/plant.
   Market use.—Fresh.
   Keeping quality.—Excellent.
   Shipping quality.—Excellent.
Resistance to diseases, insects and mites: Resistant to Phytophthora rubi, atypical of species.
Comparison with parental and known cultivars: ‘Pacific Deluxe’ differs from the proprietary female raspberry parent ‘401’ in that ‘Pacific Deluxe’ has larger fruit than ‘401. ‘Pacific Deluxe’ differs from the male raspberry parent ‘433’ in that ‘Pacific Deluxe’ is more vigorous and has a higher fruit yield than ‘433’. ‘Pacific Deluxe’ differs from the commercial variety ‘Caroline’ (U.S. Plant Pat. No. 10,412) in that ‘Pacific Deluxe’ has larger fruit with firmer flesh than ‘Caroline’.

We claim:

1. A new and distinct cultivar of raspberry plant as shown and described herein.
